(19) United States
(12) Patent Application Publication
(10) Pub. No.: US 2012/0295291 A1
(43) Pub. Date: Nov. 22, 2012

(54) Method for Determining the Risk of Clopidogrel Resistance

(75) Inventor: Andreas Rechner, Marburg (DE)

(73) Assignee: SIEMENS HEALTHCARE DIAGNOSTICS PRODUCTS GMBH, Marburg (DE)

(21) Appl. No.: 13/474,836

(22) Filed: May 18, 2012

(30) Foreign Application Priority Data
May 18, 2011 (EP) 11166477.7

(51) Int. Cl. G01N 33/86

(52) U.S. Cl. 435/13; 435/29

(57) ABSTRACT

The present invention relates to a method for identifying patients for whom there is a high probability of their not benefiting from therapy with clopidogrel. It was found that the thrombocyte activity of a patient makes it possible to establish, even before the intake of clopidogrel, whether the patient has an increased risk of clopidogrel resistance (high on-treatment platelet reactivity).

### PRIORITY STATEMENT

The present application hereby claims priority under 35 U.S.C. §119 of European Patent Application Number EP11166477.7 filed May 18, 2011, the entire contents of which are hereby incorporated herein by reference.

### FIELD OF INVENTION

The present invention is in the field of diagnosis of cardiovascular and thrombotic conditions, the goal of which is individualized therapy. In particular, the invention relates to a method for identifying patients for whom there is a high probability of their not benefiting from therapy with clopidogrel.

### BACKGROUND OF INVENTION

Clopidogrel (trade names: Plavix®, Iscover®) is a drug which inhibits blood coagulation and which is increasingly used for the routine treatment of patients suffering from an acute cardiovascular or thrombotic condition, such as myocardial infarction or stroke, or who have suffered such an event and should continue to be treated prophylactically in order to reduce the risk of a recurring thrombotic event. Clopidogrel is also administered, for example, when treating patients who are having a stent implanted into a coronary vessel, in order to avoid what are known as stent thromboses.

Clopidogrel is a P2Y12 antagonist and acts as a platelet inhibitor or thrombocyte inhibitor because it irreversibly inhibits on thrombocytes the ADP receptor P2Y12 and hence inhibits activation and aggregation of the thrombocytes. Clopidogrel is often administered in combination with acetylsalicylic acid (ASA), another thrombocyte inhibitor, since this treatment significantly reduces the risk of stroke or myocardial infarction.

However, it is known that, for numerous clopidogrel-treated patients, the desired effect of inhibition of thrombocyte activity is not achieved. This non-responsiveness to clopidogrel therapy is referred to as clopidogrel resistance or as high on-treatment platelet reactivity (HPR) (Bonello, L. et al., Consensus and future directions on the definition of high on-treatment platelet reactivity to adenosine diphosphate. J Am Coll Cardiol. 2010 Sep. 14; 56(12): 919-33). The causes of this absence of effect are unknown.

The treatment of clopidogrel-resistant patients with clopidogrel means that, for these patients, there is an undiminished risk of a renewed thrombotic event despite the therapy, and this risk persists until the clopidogrel resistance is diagnosed, for example, by means of a thrombocyte function test and the therapy is switched to another preparation. In order to provide clopidogrel-resistant patients with optimal protection from a renewed thrombotic event, said patients would have to be treated from the outset with other thrombocyte inhibitors. However, there is to date no known method with which a patient might be diagnosed as clopidogrel-resistant, without the patient actually having to be treated with clopidogrel and an absence of effect having to be established.

It is therefore desirable to have a method which makes it possible to identify those patients who are most likely to be clopidogrel-resistant. This would have the advantage that it would not be necessary at all to carry out treatment with clopidogrel and that, instead, it would be possible to administer an alternative thrombocyte inhibitor from the outset. This would, in turn, have the advantage that a clopidogrel-resistant patient can be provided with effective therapy from the outset in a targeted manner using other thrombocyte inhibitors, and as a result, there would be a decrease from the outset in the patient's risk of a renewed thrombotic event.

It is therefore an object of the present invention to provide a method which makes it possible to assess the risk of nonresponsiveness to treatment with clopidogrel for a patient for whom therapy with thrombocyte inhibitors is planned.

This object is achieved by determining the thrombocyte activity in a sample from the patient at a time at which the patient has not yet taken clopidogrel. It was found that patients with increased thrombocyte activity have an increased risk of clopidogrel resistance.

### SUMMARY OF INVENTION

The present invention therefore provides a method for determining the risk of clopidogrel resistance of a patient, comprising the following steps:
a) measuring the thrombocyte activity in a sample from the patient at a time at which the patient has not yet taken clopidogrel, and
b) comparing the measured thrombocyte activity with a statistically determined decision limit,

wherein an increased thrombocyte function beyond the decision limit indicates an increased risk of clopidogrel resistance for the patient.

### BRIEF DESCRIPTION OF THE DRAWINGS

A more complete appreciation of the present invention and many of the attendant advantages thereof will be readily understood by reference to the following detailed description when taken in conjunction with the accompanying drawings, in which:

FIG. 1 shows an embodiment of a Mosaic plot for the distribution of the response of patients to the administration of clopidogrel.

FIG. 2 shows another embodiment of Mosaic plot for the distribution of the response of patients undergoing ASA therapy to the administration of clopidogrel.

### DETAILED DESCRIPTION OF INVENTION

In the context of the present invention, the term “clopidogrel” comprises the active ingredient methyl (S)-α-(2-chlorophenyl)-6,7-dihydrothieno[3,2-c]pyridine-5(4H)-acetate and the pharmaceutically active salts thereof, for example the hydrogen sulfate, the besylate (benzenesulfonate) or the hydrochloride.

The term “clopidogrel resistance” is to be understood to mean non-responsiveness of a patient to clopidogrel therapy, or absence of effect of clopidogrel therapy in a patient. Clopidogrel resistance is present when administration of clopidogrel is not followed by attainment of the desired effect, viz. measurable inhibition of thrombocyte activity.

Indications for therapy with clopidogrel are conditions which are associated with an increased risk of thrombosis, such as cardiovascular or thrombotic conditions, for example myocardial infarction, unstable angina pectoris, ischemic stroke and peripheral arterial occlusive disease, and the implantation of vascular supports (stents) into coronary arteries.

The method according to the invention is therefore preferably suitable for determining the risk of clopidogrel resistance in patients suffering from one of the above-mentioned conditions and/or for whom stent implantation is planned and for whom antithrombotic therapy with clopidogrel should therefore be considered.

Thrombocyte activity is measured by means of a thrombocyte function test. For the determination of thrombocyte function, a range of different test formats is known, for example various thrombocyte aggregation tests. Depending on the test format, thrombocyte function can be determined in a whole blood sample or in a sample of platelet-rich plasma (PRP sample) from the patient.

In one embodiment of the method according to the invention, measurement of thrombocyte activity comprises contacting the patient's sample, preferably a whole blood sample or PRP sample, with at least one thrombocyte activator, preferably at least one thrombocyte activator from the group consisting of ADP (adenosine 5′-diphosphate), collagen, epinephrine, arachidonic acid, ristocetin and thrombin. The term “thrombocyte activator” is to be understood to mean a substance which can induce aggregation of thrombocytes. Preferably, the sample is contacted with a combination of thrombocyte activators, preferably a combination of collagen and ADP (Col/ADP) or a combination of collagen and epinephrine (Col/EPI).

In a further embodiment of the method according to the invention, measurement of thrombocyte activity comprises not only contacting the patient's sample with at least one thrombocyte activator but also contacting the patient's sample with an activator of intracellular adenylate cyclases, preferably an activator of intracellular adenylate cyclases from the group consisting of prostaglandin E1 (PGE 1), forskolin, prostaglandin I2, iloprost and cicaprost.

Measurement of thrombocyte activity can then be determined, for example, by determining the rate of thrombocyte aggregate formation in platelet-rich plasma or by determining the maximum aggregation mass of the thrombocytes, for example by means of light transmission aggregometry (Born platelet aggregation).

In a particularly preferred embodiment of the method according to the invention, a method which is insensitive to the thrombocyte-inhibiting effect of aspirin is used for measuring thrombocyte activity. Patients for whom therapy with clopidogrel is planned are almost always also treated with the thrombocyte inhibitor aspirin in order to minimize the risk of thrombosis. It was found that determining the risk of clopidogrel resistance is more precise when the thrombocyte-inhibiting effect of aspirin is blocked out by using an aspirin-insensitive method for measuring thrombocyte activity. It was further found that determining the risk of clopidogrel resistance is even more precise when a method is used which is sensitive to determination of the thrombocyte-inhibiting effect of P2Y(12) antagonists, for example clopidogrel. Such a method is described, for example, in EP-A1-1850134, paragraph 21.

In a preferred embodiment of the method according to the invention, measurement of thrombocyte activity takes place in whole blood and under flow conditions and hence in the presence of high shear forces. Such a test principle is implemented, for example, in the Platelet Function Analyzer system (PFA-100®, INNOVANCE® PFA-200, Siemens Healthcare Diagnostics GmbH, Marburg, Germany).

To simulate the flow conditions and the shear forces which prevail in relatively small arterial blood vessels, a negative pressure of about −40 mbar is generated in a special cartridge, and the citrated whole blood, which is located in a sample reservoir, flows through a capillary which has a diameter of about 100-200 μm. The capillary opens into a measuring chamber which is closed off by a partition element, for example a membrane, which contains a central capillary aperture through which the blood flows owing to the negative pressure. In most cases, the membrane, at least in the region around the aperture, contains one or more activators which induce thrombocyte aggregation, and so the blood which flows past comes into contact with the aggregation-inducing substances in the region of the aperture. The induced adhesion and aggregation of the thrombocytes results, in the region of the aperture, in the formation of a platelet plug (thrombus) which closes the membrane aperture and stops the blood flow. In this system, the time to closure of the membrane aperture is typically measured. This “closure time” (CT) correlates with the functional efficiency of the thrombocytes. A cartridge for use in a method for determining thrombocyte function by means of closure time is described, for example, in the patent document WO 97/34698. Preferred cartridges have a membrane coated with collagen (Col) and additionally either ADP or epinephrine (EPI) or a membrane coated with ADP and prostaglandin E1 (PGE1). Various partition elements and the production and use thereof are described, for example, in the patent document EP-B1-716744 or in EP-A1-1850134.

In a preferred embodiment of the method according to the invention, measurement of thrombocyte activity thus comprises conducting the sample, preferably a whole blood sample, through a capillary and subsequently through an aperture in a partition element, and measuring the time for the formation of a platelet plug at the aperture in the partition element until closure of the aperture.

Preferably, the partition element contains at least one thrombocyte activator, for example from the group consisting of ADP (adenosine 5′-diphosphate), collagen, epinephrine, arachidonic acid, ristocetin and thrombin. Particularly preferably, the partition element contains a combination of thrombocyte activators, in particular a combination of collagen and ADP (Col/ADP) or a combination of collagen and epinephrine (Col/EPI). Optionally, the partition element can additionally contain an activator of intracellular adenylate cyclases, preferably an activator of intracellular adenylate cyclases from the group consisting of prostaglandin E1 (PGE 1), forskolin, prostaglandin I2, iloprost and cicaprost.

Particularly preferably, the partition element contains ADP as thrombocyte activator, an activator of intracellular adenylate cyclases from the group consisting of prostaglandin E1 (PGE 1), forskolin, prostaglandin I2, iloprost and cicaprost, and calcium ions. By means of such a partition element, the method becomes aspirin-insensitive and, at the same time, sensitive to determination of the thrombocyte-inhibiting effect of P2Y(12) antagonists, for example clopidogrel.

In a preferred embodiment of the method according to the invention, measurement of thrombocyte activity comprises the use of a device containing the following elements:
a retention chamber for retaining the sample,
a capillary through which the sample is conducted from the retention chamber into a measuring chamber,
a measuring chamber which is divided by a partition element into two compartments, wherein the first compartment takes up the sample from the capillary,
a partition element which divides the measuring chamber into two compartments and which has an aperture through which the sample can flow from the first compartment into the second compartment.

The partition element of such a device contains at least one thrombocyte activator, preferably one of the above-described combinations of thrombocyte activators. The partition element can additionally contain one of the abovementioned activators of intracellular adenylate cyclases.

Irrespective of which test principle is used to measure the thrombocyte activity, according to the present invention, the thrombocyte activity measured in a sample from the patient is compared with a statistically determined decision limit (also known as a cut-off value).

For the determination of the decision limit, it is possible, for example, to use the statistical method of receiver operating characteristic curve (ROC curve) analysis. Based on data in which thrombocyte function was determined in the same patient both before and after the intake of clopidogrel, it is possible with this method to calculate an optimal decision limit for a two-class classification which enables patients having an increased risk of clopidogrel resistance to be distinguished from patients having a distinctly lower risk of clopidogrel resistance. For this purpose, the results from at least 60 patients, even better 120 or more patients, should be included in the calculation.

“Increased risk of clopidogrel resistance” is to be understood to mean that the probability of clopidogrel having no effect in a patient is increased by about 2- to 4-fold, with more than half of patients being nonresponsive to clopiodgrel in the case of an increased risk. In the case of a lower risk, this number is only about a quarter to a fifth of patients.

### DESCRIPTION OF THE FIGURES

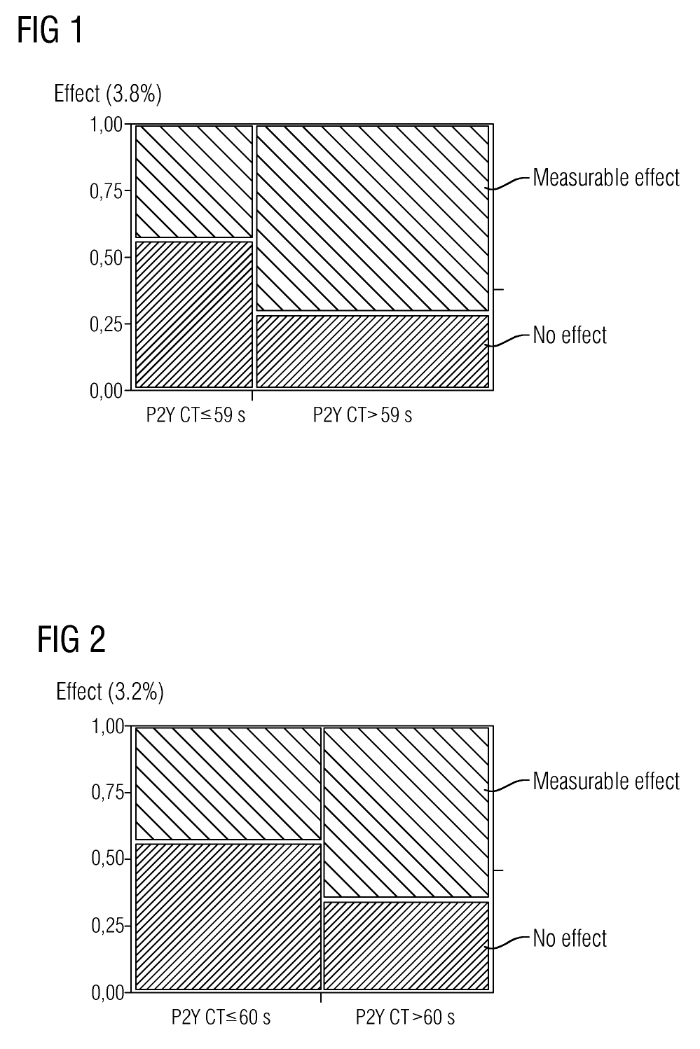

FIG. 1

Mosaic plot for the distribution of the response of patients to the administration of clopidogrel (“Effect”) in the case of INNOVANCE PFA P2Y closure times (“P2Y CT”) of ≦59 s and >59 s when using 3.8% strength citrated whole blood prior to the intake of clopidogrel. The width and height of the tiles indicate the relative proportion of the two classes with respect to the total population.

FIG. 2

Mosaic plot for the distribution of the response of patients undergoing ASA therapy to the administration of clopidogrel (“Effect”) in the case of INNOVANCE PFA P2Y closure times (“P2Y CT”) of ≦60 s and >60 s when using 3.2% strength citrated whole blood prior to the intake of clopidogrel. The width and height of the tiles indicate the relative proportion of the two classes with respect to the total population.

### EXAMPLES

### Study Design

Blood was withdrawn from 143 patients suffering from cardiovascular conditions who had been selected for surgical insertion of a stent, with blood being withdrawn before and 6 to hours after they had taken, as is customary for this procedure, 300 or 600 mg of clopidogrel (loading dose).

The patients had neither medical records nor laboratory results to indicate thrombocyte dysfunction caused by intrinsic thrombocyte defects, by VWF defects (VWD) or by the intake of thrombocyte aggregation inhibitors other than acetylsalicylic acid. The majority of the patients were already taking 81 or 100 mg of acetylsalicylic acid (ASA) each day at the time of blood withdrawal.

Thrombocyte activity in the patients' samples was determined using the Platelet Function Analyzer system (PFA-100®, Siemens Healthcare Diagnostics Products GmbH, Marburg, Germany).

Using the PFA system, primary hemostasis is measured in whole blood samples under flow conditions and hence in the presence of high shear forces. As a measure of thrombocyte function, the time to closure of a membrane aperture in a special cartridge (closure time) is measured in this system. Closure time is inversely proportional to thrombocyte function, i.e., the longer the closure time, the lower the thrombocyte activity.

Cartridges which have different types of membrane enable different functions of thrombocytes to be determined. Cartridges were used which were provided with a membrane having a collagen (Col) and ADP coating (Col/ADP; aspirin-insensitive), or having a collagen (Col) and epinephrine (EPI) coating (Col/EPI; aspirin-sensitive), or having an ADP, prostaglandin E1 and calcium ion coating (INNOVANCE® PFA P2Y; aspirin-insensitive and sensitive to the thrombocyte-inhibiting effect of P2Y(12) antagonists).

For the three different types of cartridges, thresholds (cut-offs) for the closure time (CT) (in seconds) had been determined beforehand, which allow differentiation between normal thrombocyte function and reduced thrombocyte function. Samples with reduced thrombocyte function have a prolonged closure time which is above the cut-off. Samples which, after the administration of clopidogrel, had a closure time above the cartridge-specific cut-off exhibited the desired effect of clopidogrel therapy, viz. measurable inhibition of thrombocyte activity. Samples which had, by contrast, a closure time below the cartridge-specific cut-off after the administration of clopidogrel exhibited absence of effect of clopidogrel therapy, i.e., clopidogrel resistance (or high on-treatment platelet reactivity).

The cut-offs for the closure times of the different types of cartridges (in seconds) for differentiating normal thrombocyte function from reduced thrombocyte function are shown in table 1.

TABLE 1
Cut-offs of the three PFA cartridges type-specific closure
times for determining reduced thrombocyte function

| | Col/EPI | Col/ADP | INNOVANCE PFA P2Y |
|---|---|---|---|
| Cut-off | 165 s | 119 s | 106 s |

The closure time measurements with INNOVANCE PFA P2Y cartridges were carried out both with 3.2% and with 3.8% citrated whole blood, whereas the Col/EPI and Col/ADP closure times were measured only in 3.2% citrated whole blood.

Altogether, by means of the closure time measurements using INNOVANCE PFA P2Y cartridges following intake of clopidogrel, it was found that 45.8% (3.2% citrated whole blood) or 37.5% (3.8% citrated whole blood) of the patients studied were clopidogrel-resistant.

It was observed that the group of patients for whom it was not possible to achieve any measurable inhibition of thrombocyte activity by the clopidogrel therapy and who were thus afflicted with clopidogrel resistance, already exhibited prior to the intake of clopidogrel significantly increased thrombocyte function (in the form of short closure times) compared to the other patient group who responded to the clopidogrel therapy.

Determination of abnormally increased thrombocyte function prior to the intake of clopidogrel thus allows an increased risk of clopidogrel resistance to be predicted.

### Determination of the Decision Limit

In order to be able to differentiate abnormally increased thrombocyte function from normal thrombocyte function prior to the intake of clopidogrel and to thus determine an increased risk of clopidogrel resistance, the results from 143 patients were used for the calculation of an optimal decision limit. For each type of cartridge and, in the case of INNOVANCE PFA P2Y cartridges, additionally for each citrate concentration used a separate decision limit was calculated. The classification variable used for the ROC curve analysis was the response to clopidogrel in the corresponding citrated whole blood.

The optimal decision limits thus determined are presented in table 2.

TABLE 2
Optimal decision limits (PFA cartridge type-specific closure times)
for the prediction of increased risk of clopidogrel resistance

| | INNOVANCE | INNOVANCE | | |
|---|---|---|---|---|
| | PFA P2Y | PFA P2Y | Col/EPI | Col/ADP |
| | (with 3.2% | (with 3.8% | (with 3.2% | (with 3.2% |
| | citrated | citrated | citrated | citrated |
| | blood) | blood) | blood) | blood) |
| Decision | ≦60 s | ≦59 s | ≦140 s | ≦91 s |
| limit | | | | |

### Predictive Value of Short Closure Times

If the INNOVANCE PFA P2Y closure time of a patient in 3.8% citrated whole blood prior to the intake of clopidogrel is less than or equal to 59 seconds, the relative risk of said patient not responding to clopidogrel is 98% higher than for patients with closure times greater than 59 seconds (relative risk: 1.98; p<0.002; Fisher's exact test). The distribution of the results is shown in FIG. 1.

If the INNOVANCE PFA P2Y closure time of a patient in 3.2% citrated whole blood prior to the intake of clopidogrel is less than or equal to 60 seconds, the relative risk of said patient not responding to clopidogrel is 61% higher than for patients with closure times greater than 60 seconds (relative risk: 1.61; p<0.02; Fisher's exact test). The distribution of the results from patients undergoing ASA therapy is shown in FIG. 2.

If the Col/EPI closure time of a patient in 3.2% citrated whole blood prior to the intake of clopidogrel is less than or equal to 140 seconds, the relative risk of said patient not responding to clopidogrel is 78% higher than for patients with closure times greater than 140 seconds (relative risk: 1.78; p<0.004, Fisher's exact test).

Interestingly, in the case of INNOVANCE PFA P2Y measurement after clopidogrel intake in 3.8% citrated whole blood, the relative risk of patients not responding to clopidogrel is even 164% higher if the Col/EPI closure time measured prior to the intake of clopidogrel in 3.2% citrated whole blood is 140 seconds (relative risk: 2.64; p<0.0001; Fishers exact test).

If the Col/ADP closure time of a patient undergoing ASA therapy in 3.2% citrated whole blood prior to the intake of clopidogrel is less than or equal to 91 seconds, the relative risk of said patient not responding to clopidogrel is 47% higher than for patients with closure times greater than 91 seconds (relative risk: 1.47; p=0.06; Fisher's exact test).

The invention claimed is:

1. A method for determining the risk of clopidogrel resistance of a patient, comprising the following steps:
a) measuring the thrombocyte activity in a sample from the patient at a time at which the patient has not yet taken clopidogrel, and
b) comparing the measured thrombocyte activity with a statistically determined decision limit,
wherein an increased thrombocyte function beyond the decision limit indicates an increased risk of clopidogrel resistance for the patient.

2. The method as claimed in claim 1, wherein the patient is suffering from a cardiovascular or thrombotic condition and/or has been selected for stent implantation.

3. The method as claimed in claim 1, wherein the measurement of thrombocyte activity in step a) comprises the following step:
contacting the sample with at least one thrombocyte activator,
preferably with at least one thrombocyte activator from the group consisting of ADP, collagen, epinephrine, arachidonic acid, ristocetin and thrombin.

4. The method as claimed in claim 3, wherein the sample is contacted with a combination of thrombocyte activators, preferably with collagen and ADP or with collagen and epinephrine.

5. The method as claimed in claim 3, wherein the sample is additionally contacted with an activator of intracellular adenylate cyclases,
preferably from the group consisting of prostaglandin E1 (PGE 1), forskolin, prostaglandin I2, iloprost and cicaprost.

6. The method as claimed in claim 1, wherein the measurement of thrombocyte activity in step a) comprises the following steps:
conducting the sample through a capillary and subsequently through an aperture in a partition element, and
measuring the time for the formation of a platelet plug at the aperture in the partition element until closure of the aperture.

7. The method as claimed in claim 6, wherein the partition element contains at least one thrombocyte activator, preferably a combination of thrombocyte activators.

8. The method as claimed in claim 1, wherein the measurement of thrombocyte activity in step a) uses a method which is insensitive to the thrombocyte-inhibiting effect of aspirin.

9. The method as claimed in claim 8, wherein the method is additionally sensitive to the thrombocyte-inhibiting effect of P2Y(12) antagonists.

10. The method as claimed in claim 1, wherein the sample is whole blood or platelet-rich plasma.

11. The method as claimed in claim 1, wherein the measurement of thrombocyte activity in step a) comprises the use of a device containing the following elements:
a retention chamber for retaining the sample,
a capillary through which the sample is conducted from the retention chamber into a measuring chamber,
a measuring chamber which is divided by a partition element into two compartments, wherein the first compartment takes up the sample from the capillary,
a partition element which divides the measuring chamber into two compartments and which has an aperture through which the sample can flow from the first compartment into the second compartment,
wherein the partition element contains at least one thrombocyte activator, preferably a combination of thrombocyte activators.

12. The method as claimed in claim 11, wherein the partition element contains ADP as thrombocyte activator, an activator of intracellular adenylate cyclases from the group consisting of prostaglandin E1 (PGE 1), forskolin, prostaglandin I2, iloprost and cicaprost, and calcium ions.
